(19) United States
(12) Patent Application Publication
(10) Pub. No.: US 2007/0060949 A1
(43) Pub. Date: Mar. 15, 2007

(54) Inflatable tourniquet to aid in establishing intravenous access and method thereof

(76) Inventors: Ammie D. Curry, Mount Clare, WV (US); Steven T. Curry, Mount Clare, WV (US)

Correspondence Address: Tina L. Cunningham, 100 Washington St., Bentleyville, PA, 15314, US

(21) Appl. No.: 11/222,536

(22) Filed: Sep. 9, 2005

(51) Int. Cl. A61B 17/00

(52) U.S. Cl. 606/201

(57) ABSTRACT

A device to aid in establishing intravenous access by means of a sequential trapping and displacing of the venous blood in a top to bottom action down an extremity, resulting in a localized increase in the diameter of the veins, due to increased, trapped blood volume. The resulting distention of the vessels produces a stabilizing effect, thereby decreasing vascular mobility and rolling. The combination of vessel distention and stabilization greatly facilitates the obtaining of a patent I.V. The sequential trapping and milking of venous blood toward the extremity is accomplished via a specialized inflatable bladder.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Many medical procedures and almost all emergency medical situations require a patent intravenous access for administration of fluids, medications, nutrients, etc. Often, I.V. access is extremely difficult to obtain. This is especially so in the elderly, the obese, “shocky” patients, and so forth. Therefore, the purpose of this device is to facilitate rapid, effective and efficient I.V. access for all patients.

There are current patents on devices such as tourniquets and compressors which are used to encircle a limb for a number of purposes (including for the purpose of inserting an I.V.). However, none of these devices act in the manner of the current apparatus where the device is removable once the I.V. has been inserted and uses the sequential filling of air bladders to displace the blood from the upper part of the extremity to the lower portion.

### BRIEF SUMMARY OF THE INVENTION

According to the invention, a specialized elastomeric bladder is utilized to provide for blood movement in the extremity. This provides for more efficient access to patient's veins for the insertion of an I.V. This elastomeric bladder would be composed of a series of chambers with air flow restrictions between the chambers. Alternatively, the bladder would contain a plurality of separate and individual chambers. The bladder could also contain exhaust valves between the chambers to allow the air to escape and release pressure. The bladder is then removably secured around the limb and inflated. The design is such that the chambers inflate proximal to distal (top to bottom) sequentially. For example, in a bladder with a series of chambers, air flow begins in the proximal chamber. Due to the interconnectivity of the chambers, the proximal chamber will inflate and then the lower (distal) chambers will inflate. In a bladder containing separate individual chambers, the air flow will begin in the proximal chamber. Then the lower chambers will be inflated using air flow restriction devices, separate tubing, etc., between the chambers. This results in a milking of the venous blood into the extremity which causes a distention and stabilization of the veins.

The current invention is advantageous over the prior art in that the device is removable once the I.V. has been inserted. This allows for easy use in any medical situation. Further, the bladder itself is the apparatus which causes the blood movement, not a strap or any other kind of implement which could pinch or place uneven pressure on the limb. Also, the bladder chambers used in the current device are not separate ring shaped chambers, but instead are sequential and separated by air flow restriction devices, separate tubing, etc.

### BRIEF DESCRIPTION OF THE SEVERAL VIEWS OF THE DRAWING

FIG. 1 is one embodiment of the completed apparatus including a mechanism for securing the apparatus around a limb and a protective cover for the bladder.

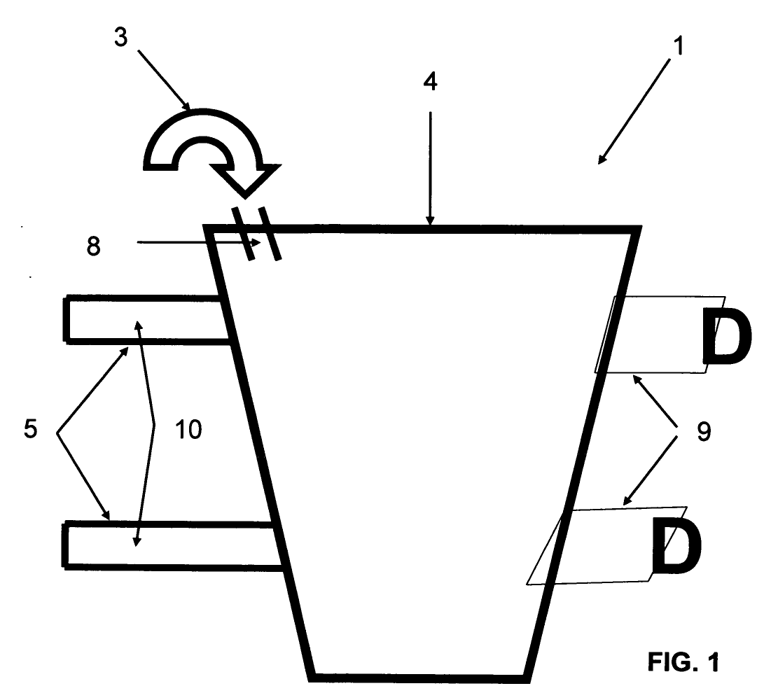

FIG. 2 is a cross section diagram of the elastomeric bladder having two chamber, showing the sequential nature of the chambers as well as the air flow restrictions.

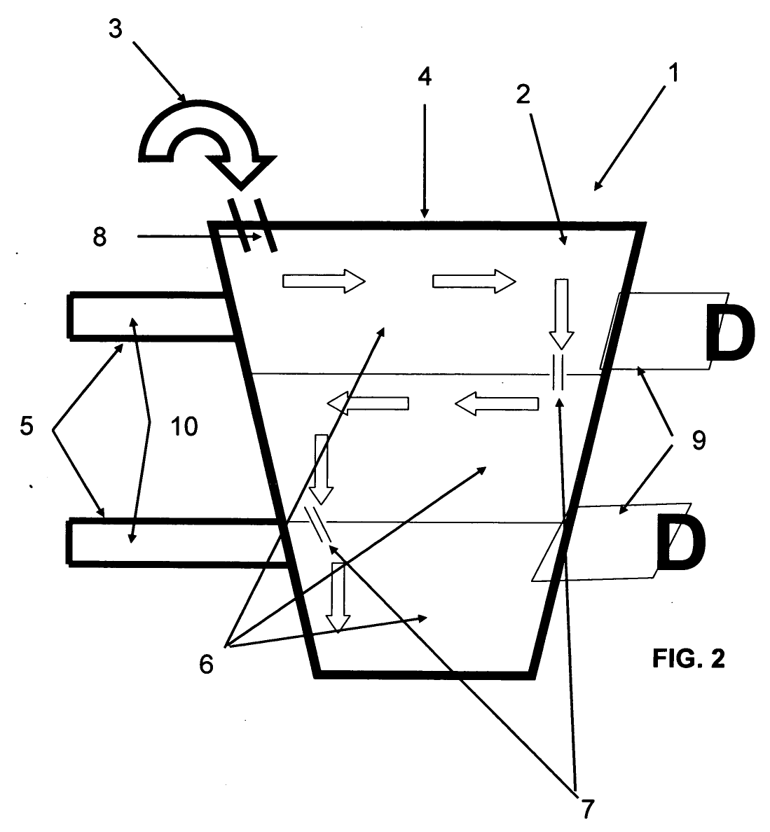

FIG. 3 is a cross section diagram of the elastomeric bladder having a plurality of sequential chambers.

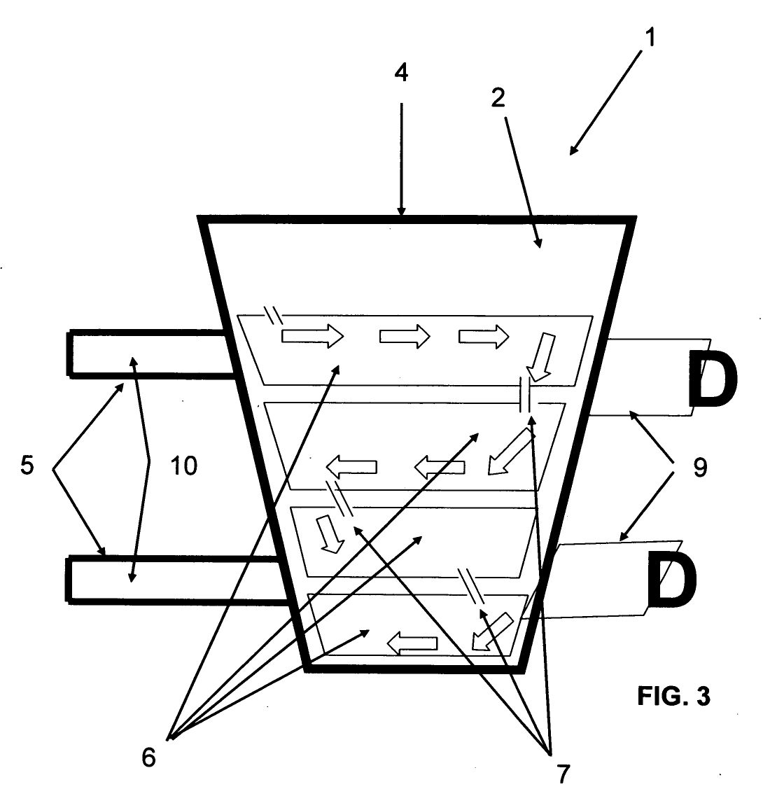

FIG. 4 is a cross section diagram of the elastomeric bladder having a plurality of separate and individual chambers as well as exhaust valves.

### DETAILED DESCRIPTION OF THE INVENTION

The embodiment of the inflatable tourniquet 1 comprises an elastomeric bladder 2 with a plurality of sequentially positioned air flow chambers 6 that are within the bladder 2 along with air flow restriction devices 7 positioned between the chambers. In order to inflate the bladder 2 an opening for air flow 8 is present at the top of the bladder 2. Air is entered into the bladder through any kind of air pump device 3, including a standard bulb pump or a battery operated pump. There can also be a protective cover 4 which is positioned around the elastomeric bladder 2. The inflatable tourniquet will also have means for securing the inflatable tourniquet 1 to a limb including, but not limited to, a plurality of straps 5 on one side of the bladder 2 and a plurality of receiving members 9 on the other side of the bladder. The straps 5 will then be secured upon themselves through any standard means of securing including Velcro®, snaps, zippers, buttons or any similar fastening device 10. Any number of methods of securing the inflatable tourniquet to the limb could be used including the strap 5 and receiving member 9 system described above. Other methods of securing could include ties on both sides of the bladder. Similarly, straps could be present on both sides of the bladder which are then secured together using a standard fastening means such as Velcro®, snaps or buttons.

The elastomeric bladder 2 could be comprised of any type of material suitable for retaining air and applying pressure, including, but not limited to: rubber, latex, silicone, or plastic. The size of the elastomeric bladder 2 and therefore the inflatable tourniquet 1 is variable and can be adapted for use on different limbs and in different sized patients. The number of chambers 6 present in the elastomeric bladder 2 is variable and can be altered depending on the size bladder needed.

The air flow restrictions 7 present in the elastomeric bladder 2 are also variable and depend on the number of air chambers 6 present in the elastomeric bladder 2. Air flow restriction can be accomplished by any of a number of different devices including valves, tubes or small diameter airspace.

The cover 4 placed over the elastomeric bladder 2 is unnecessary and a single laminated piece could be used instead. However, if a cover 4 is used, it could be made of any type of sturdy protective material, including but not limited to nylon, cotton, synthetic fiber, etc. Any decorative emblem could then be placed on this protective cover 4 in order to identify the product.

The air flow opening 8 could be positioned either from the left, right or center of the inflatable tourniquet 1. Further, the device used for providing the air 3 could be any standard device known, including a handheld air pump as well as a battery operated air pump.

Any additional number of methods of securing the inflatable tourniquet to the limb could be used including the strap 5 and receiving member 9 system described above. Other methods of securing could include ties on both sides of the bladder. Similarly, straps could be present on both sides of the bladder which are then secured together using a standard fastening means such as Velcro®, snaps or buttons.

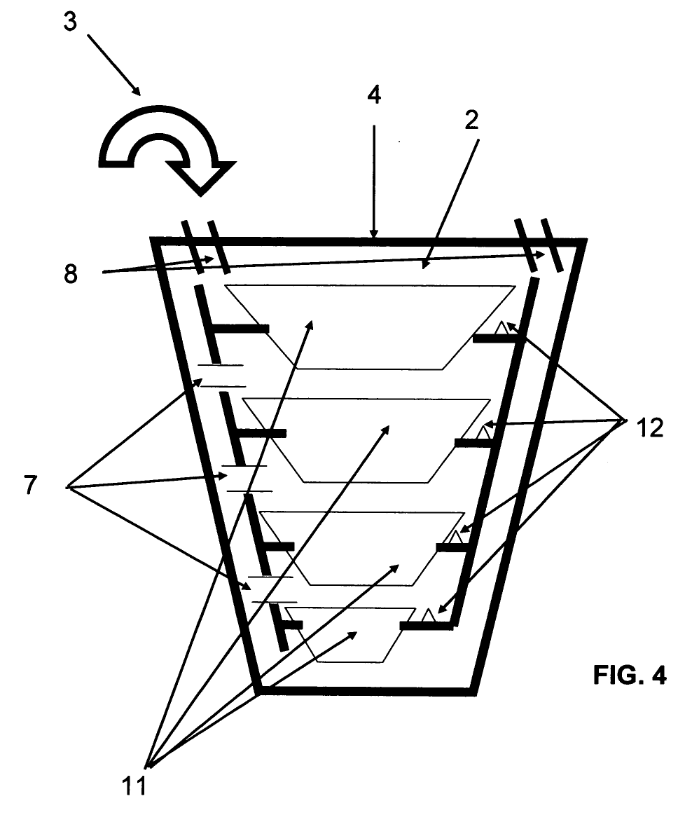

As seen in FIG. 4, the elastomeric bladder 2 could also contain a plurality of separate and independent inflatable chambers 11. Therefore, instead of the chambers being sequential and interconnected, the chambers could be separate and independent. Similarly, the bladder 2 would be inflated by using an opening for air flow 8 that is present at the top of the bladder 2. Air is entered into the bladder through any kind of air pump device 3, including a standard bulb pump or a battery operated pump. The air will inflate the lower (distal) chambers of the bladder 2 using air flow restriction devices 7 between the proximal and distal chambers. The release of the air and pressure in the bladder can be released through the use of exhaust valves 12 and an air flow opening 8.

These terms and specifications serve to describe the invention by example and not to limit the invention. It is expected that others will perceive differences, which, while differing from the forgoing, do not depart from the scope of the invention herein described and claimed. In particular, any of the functional elements described herein may be replaced by any other known element having an equivalent function.

What is claimed is:

1. An inflatable tourniquet to aid in establishing intravenous access comprising:
An elastomeric bladder comprising an opening within said elastomeric bladder for inflation and deflation of said elastomeric bladder and wherein said elastomeric bladder contains a plurality of air flow chambers;
A plurality of air flow restriction devices between said air flow chambers;
A means of inflating said elastomeric bladder; and
A means for securing said elastomeric bladder to a patient's limb.

2. The inflatable tourniquet to aid in establishing intravenous access of claim 1 wherein said plurality of air flow chambers are positioned sequentially within said elastomeric bladder.

3. The inflatable tourniquet to aid in establishing intravenous access of claim 1 wherein said plurality of air flow chambers are separate and individual and positioned parallel within said elastomeric bladder.

4. The inflatable tourniquet to aid in establishing intravenous access of claim 1 wherein said inflatable tourniquet further comprises a plurality of exhaust valves between said air flow chambers.

5. The inflatable tourniquet to aid in establishing intravenous access of claim 1 wherein said elastomeric bladder further comprises a protective cover positioned around said elastomeric bladder.

6. The inflatable tourniquet to aid in establishing intravenous access of claim 2 wherein said elastomeric bladder further comprises a protective cover positioned around said elastomeric bladder.

7. The inflatable tourniquet to aid in establishing intravenous access of claim 3 wherein said elastomeric bladder further comprises a protective cover positioned around said elastomeric bladder.

8. A method of establishing IV access in a patient comprised of the trapping and displacement of venous blood in a limb wherein said trapping a displacement of venous blood is further comprised of:
Attaching an inflatable tourniquet;
Inflating said inflatable tourniquet sequentially; and
Inserting an IV.

9. The method of establishing IV access in a patient of claim 8 further comprising the removal of said inflatable tourniquet after said inserting of an IV.

10. The method of establishing IV access in a patient of claim 8 wherein said inflatable tourniquet further comprises:
An elastomeric bladder comprising an opening within said elastomeric bladder for inflation and deflation of said elastomeric bladder and wherein said elastomeric bladder contains a plurality of air flaw chambers;
A plurality of air flow restriction devices between said air flow chambers;
A means of inflating said elastomeric bladder; and
A means for securing said elastomeric bladder to a patient's limb.

11. The method of establishing IV access in a patient of claim 8 wherein said plurality of air flow chambers are positioned sequentially within said elastomeric bladder.

12. The method of establishing IV access in a patient of claim 8 wherein said elastomeric bladder further comprises a protective cover positioned around said elastomeric bladder.

13. An elastomeric bladder comprising:
An opening for inflation and deflation of said elastomeric bladder;
A plurality of air flow chambers;
A plurality of air flow restriction devices between said air flow chambers;
A means for securing said elastomeric bladder to a patient's limb; and
A means for inflating said elastomeric bladder.

14. The elastomeric bladder of claim 13 wherein said plurality of air flow chambers are positioned sequentially within said elastomeric bladder.

15. The elastomeric bladder of claim 13 wherein said plurality of air flow chambers are separate and individual and positioned parallel within said elastomeric bladder.

16. The elastomeric bladder of claim 13 wherein said elastomeric bladder further comprises a plurality of exhaust valves between said air flow chambers.

17. The elastomeric bladder of claim 13 wherein said elastomeric bladder further comprises a protective cover positioned around said elastomeric bladder.

18. The elastomeric bladder of claim 14 wherein said elastomeric bladder further comprises a protective cover positioned around said elastomeric bladder.

19. The elastomeric bladder of claim 15 wherein said elastomeric bladder further comprises a protective cover positioned around said elastomeric bladder.
